(19) United States
(12) Patent Application Publication
(10) Pub. No.: US 2015/0305602 A1
(43) Pub. Date: Oct. 29, 2015

(54) INTEGRATED MEDICAL IMAGING SYSTEM

(71) Applicant: Calcula Technologies, Inc., San Francisco, CA (US)

(72) Inventors: David GAL, San Francisco, CA (US); Raymond Arthur BONNEAU, San Francisco, CA (US)

(21) Appl. No.: 14/561,364

(22) Filed: Dec. 5, 2014

Related U.S. Application Data
(60) Provisional application No. 61/983,419, filed on Apr. 23, 2014.

(51) Int. Cl. A61B 1/04; A61B 1/307; A61B 1/00; A61B 1/07

(52) U.S. Cl. CPC A61B 1/042; A61B 1/07; A61B 1/307; A61B 1/00121

(57) ABSTRACT

A fiber optic camera system may include a fiber optic camera and a video processing console. The camera may include an elongate sheath having a proximal end and a distal end, and the sheath may contain one or more illumination optical fibers and an imaging bundle having at least one fiber optic clad and multiple fiber optic cores. The camera may further include a camera body fixedly attached to the proximal end of the elongate sheath, and the camera body may contain an imaging sensor optically coupled to a proximal end of the imaging bundle and configured to generate image data and an illumination source optically coupled to proximal ends of the illumination fibers. In some embodiments, the camera body has no connection member for connecting a secondary illumination source to the camera.

### CROSS-REFERENCE TO RELATED APPLICATIONS

This application claims priority to U.S. Provisional Patent Application No. 61/983,419, filed on Apr. 23, 2014, the disclosure of which is hereby fully incorporated by reference.

### FIELD OF THE DISCLOSURE

The present disclosure is related to visualization devices for medical and/or surgical procedures. More specifically, the disclosure is related to flexible, elongate cameras for visualizing within a human or animal body.

### BACKGROUND

Visualization of tissues, structures and tools in medical practice is often critical to a successful clinical outcome. During traditional open surgeries and procedures, this was relatively trivial—the practitioner simply looked into the body. With the advent of minimally invasive and endoscopic procedures, however, advances in visualization have become necessary to properly view the surgical field. To that end, advances in visualization technology have paralleled the miniaturization of surgical tools and techniques.

The primary way to directly visualize an endoscopic procedure is to insert a camera into the field and observe an image acquired by the camera on a monitor. The two most commonly used types of cameras for visualizing within a human or animal body are “chip-on-stick” and fiber optic cameras. Chip-on-stick refers to the use of a CMOS (complementary metal oxide semiconductor) or CCD (charge-coupled device) sensor at the distal end of a medical instrument. Sensor 24 converts the image (light) signal into an electrical signal, which is transmitted to the proximal end of the medical instrument. Fiber optic cameras use optical fibers (usually several thousand) to transmit light from the scene of interest via the principle of total internal reflection to a sensor or eyepiece on the proximal end of the medical device. Each fiber in the bundle is effectively a “pixel” in a spatially sampled image. Typically an eyepiece is attached at the proximal end of the medical device, so the user can see the light each fiber carries down the instrument.

Fiber cameras currently have a larger market share than chip-on-stick technology. This is due to the relative nascency of chip-on-stick technology. Generally speaking, chip-on-stick devices provide a higher quality image and a theoretical lower price point but are typically larger than fiber based solutions. Fiber optic solutions are generally required when a small camera cross-sectional is desired.

Direct visualization systems for medical applications (both chip on stick and fiber) are generally packaged into large, general purpose medical devices that facilitate the delivery of other application specific devices to particular areas of the body. Typically, the application specific tools are disposable, and the guiding endoscope is more expensive, reusable capital equipment. In urology, for example, a general purpose reusable flexible ureteroscope provides imaging and navigation of a working channel, in which disposable baskets, graspers, lasers, and the like are guided to the location of interest.

The imaging system of a typical fiber optic based endoscope is constructed with an eyepiece optically coupled to an imaging fiber optic bundle and a light post optically coupled to illumination fibers. The imaging bundle is either comprised of several discrete fibers, each with its own fiber optic core and fiber optic clad bundled together, or a single fiber optic cable containing multiple fiber optic cores sharing a common fiber optic clad. A light box placed on an endoscopic tower containing a high power illumination source is connected to the light post by a light cable—a long bundle of optical fibers, which transmit light from the source to the distal end of the endoscope. Typical light boxes are constructed with Xenon lamps and consume on the order of hundreds of Watts of power. The user can either look through the eyepiece or attach a camera head to the eyepiece, which images the scene. These “clip-on” cameras typically transmit image information to a video-processing console, which sits on the endoscopic tower via a multi-conductor cable. The console ultimately displays the video information to a monitor, where it is easily observed. Naturally, the latter visualization option has mostly obsoleted the use of an eyepiece. The general purpose, fiber based endoscope requires at least two bulky cables, one for the clip-on camera and one for the illumination source. These cables and accessories add substantial weight and bulk to the system, which degrade the ergonomic and user experience.

Fiber based imaging systems are usually delicate and malfunction after repeated use and sterilization. There are several “weak points” in the system, which can cause failure: illumination fibers crack, imaging fibers break, fibers in the light cable break, clip-on cameras fall, and lenses shift out of focus. Because the imaging system is a part of the endoscope a failure in the imaging system renders the endoscope useless, and a failure in the endoscope (broken pull wires, etc.) renders the imaging system useless. The repair costs of endoscopes and their fiber based imaging systems are extremely high and a significant pain point for medical facilities.

In summary, currently available, medical grade, fiber-based imaging systems are generally bulky, cumbersome, expensive, and include several weak points. Therefore, it would be advantageous to have improved medical imaging systems.

### BRIEF SUMMARY

As mentioned above, the general-purpose endoscope is effectively a delivery mechanism for specialized functional tools. Many medical procedures and tools that may benefit from direct visualization are incompatible with the use of any currently available endoscope. Difficult uretheral catheterizations, for example, may benefit from direct visualization, but Foley catheters may be too large for the working channel of the typical endoscope. There are other medical procedures in which endoscopes are used, but for which the endoscope itself results in an overall larger instrument diameter than necessary. Extracting ureteral stones, for example, does not necessarily require all the features of a typical ureteroscope but would benefit from a scope with a small outer diameter. Imaging the fallopian tubes, sinuses, gastrointestinal tract, and lungs are all cases were it may be advantageous to use an imaging device with a smaller diameter than that of a traditional endoscope.

The present disclosure describes a fiber-based, medical imaging system, which is separate from any particular medical device and more robust than typical currently available systems. In some embodiments, the system is fully integrated, meaning that the fiber, camera and light source are combined a single unit. In alternative embodiments, the system may include a fiber bundle and a mating feature for helping couple the fiber bundle with other disposable or reusable medical devices. In these embodiments, it may be possible to mate the camera and the medical device without guiding the device through the working channel of a camera, but rather by guiding the camera through the device. These embodiments may allow many existing medical devices to take advantage of direct visualization. Additionally, these embodiments may simplify new device design, since devices need not be designed around the dimensions of an existing endoscope working channel, but rather may simply include an extremely small channel to allow for passage of the disclosed imaging system. This allows the medical devices themselves to have any of a number of desirable outer diameters for performing various procedures.

In one aspect of the present invention, a fiber optic camera system may include a fiber optic camera and a video processing console coupled with the camera. The camera may include an elongate sheath having a proximal end and a distal end, and the sheath may contain one or more illumination optical fibers and an imaging bundle comprising at least one fiber optic clad and multiple fiber optic cores. The camera may further include a camera body fixedly attached to the proximal end of the elongate sheath, and the camera body may contain an imaging sensor optically coupled to a proximal end of the imaging bundle and configured to generate image data and an illumination source optically coupled to proximal ends of the illumination fibers. The video processing console may be coupled wirelessly or via a cord with the camera body and may be configured to process the image data from the imaging sensor to generate at least one output signal. In some embodiments, the camera body has no connection member for connecting a secondary illumination source to the camera.

Some embodiments of the system may further include a cable for connecting the camera body with the video processing console, and connection between the camera body and the video processing console is achieved solely via the cable. In some embodiments, the sheath may include polytetrafluoroethylene. In some embodiments, the sheath may have a reinforced configuration, a braided configuration and/or a coiled configuration. Optionally, the camera body may further contain a data serializer, and the console may include a data deserializer. In such an embodiment, the imaging sensor is configured to output image data using multiple parallel signals, the data serializer is configured to convert the multiple parallel signals into at least one pair of differential signals, and the deserializer is configured to convert the at least one pair of differential signals into multiple parallel signals.

In some embodiments, the illumination fibers include cores and clads, and distal ends of the cores of the illumination fibers have a total surface area of less than about 0.000045 square-inches. In some embodiments, the one or more illumination optical fibers comprise about 20 to about 40 illumination fibers. In some embodiments, the imaging sensor has a responsiveness of at least 4.8V/lux-s. In some embodiments, the sheath has an outer diameter of no greater than approximately 0.7 millimeters.

Optionally, the system may further include a medical device having a lumen capable of removably receiving the sheath. In one embodiment, the medical device is configured for use in a urinary tract of a human or animal subject. In some embodiments, a proximal end of the medical device includes a mating feature configured to mate with a corresponding mating feature on the camera body. Optionally, the mating feature and the corresponding mating feature may include locking features for removably coupling the medical device with the camera body. In one embodiment, the locking features allow a connection between the mating feature and the corresponding mating feature to be slidably adjusted to ensure alignment within approximately 0.5 mm. In some embodiments, the camera body may further include a mechanism configured to identify the medical device and determine whether the medical device is compatible with the camera.

In some embodiments, the camera body further contains a one or more proximal lenses. In some embodiments, the camera body further includes a thermal bridge that thermally couples the illumination source to the camera body. In some embodiments, the camera body is substantially hermetically sealed. In some embodiments, the camera body further contains a nonvolatile memory module coupled with the console. In some embodiments, a single control bus is electrically coupled to the imaging sensor, a nonvolatile memory module, and/or a circuit for controlling the illumination source. In some embodiments, the system may further include a video monitor for connecting with the video processing console, where the output signal from the video processing console drives the video monitor. In some embodiments, the illumination source includes a light emitting diode.

In another aspect, a medical fiber optic camera may include: an elongate sheath having a proximal end, a distal end, and an outer diameter of no more than approximately 0.7 millimeters; one or more illumination optical fibers disposed within the sheath; an imaging bundle disposed within the sheath and comprising at least one fiber optic clad and multiple fiber optic cores; a camera body fixedly attached to the proximal end of the elongate sheath and having no connector for connecting a secondary light source to the camera; an imaging sensor housed in the camera body, optically coupled to a proximal end of the imaging bundle and configured to generate image data; and a light-emitting diode housed in the camera body and optically coupled to proximal ends of the illumination fibers.

In some embodiments, the imaging sensor is further configured to process the image data to generate an output signal. In some embodiments, the camera body further contains a nonvolatile memory module. In some embodiments, a single control bus is electrically coupled to the imaging sensor, a nonvolatile memory module, and/or circuitry controlling the illumination source.

In some embodiments, the sheath is configured to be inserted into a lumen of a medical device. In some embodiments, the medical device is configured for use in urinary tract of a human or animal subject. Examples of medical devices include, but are not limited to, a urinary stone removal catheter device, a guide catheter, other catheter devices, a steerable sheath, an endoscope, and an access sheath. In some embodiments, the camera body comprises a mating feature configured to mate with a corresponding mating feature on a proximal end of the medical device. In some embodiments, the outer diameter of the sheath is less than about 0.6 millimeters.

In another aspect, a method of imaging a scene of interest in a human or animal subject may involve: advancing an elongate sheath of a fiber optic camera, containing one or more illumination optical fibers and an imaging fiber bundle, into a human or animal subject to position a distal end of the sheath near a scene of interest, wherein the sheath has an outer diameter of no more than approximately 0.7 millimeters; illuminating the scene of interest with the one or more illumination optical fibers, wherein the proximal ends of the illumination fibers are coupled with a light-emitting diode in a camera body fixedly attached to a proximal end of the sheath; capturing light information with an imaging sensor in the camera body coupled with a proximal end of the imaging fiber bundle; converting the light information into image data with the imaging sensor; and transmitting the image data from the imaging sensor through a single connection to a video processing console or a video display monitor.

In some embodiments, transmitting the image data may involve transmitting the signal to the video processing console, and the method may further involve processing the image data using the video processing console to generate an output and providing the output for display on the video display monitor. Optionally, the method may also involve serializing at least part of the image data via a data serializer in the camera body and deserializing the image data via a deserializer in the console. In some embodiments, the method may further involve controlling a parameter of the imaging sensor via the console. Such embodiments may optionally also involve configuring the parameter of the imaging sensor based on a camera parameter. In such embodiments the parameter of the imaging sensor may include, but is not limited to, gain, exposure, exposure time, gamma correction, frame rate, output image size, and/or region of interest.

Optionally, the method may also include configuring a parameter of the illumination source via the console. In some embodiments, the parameter of the illumination source is LED drive current. The method may also further include: determining, using a non-volatile memory in the camera body, a number of times the camera has been used; updating the number of times after each usage of the camera; and providing an alert when the number of times exceeds a predetermined maximum number of times. The method may also further include increasing exposure by reducing an area of readout of the imaging sensor to a region of interest smaller than a total area of the imaging sensor to increase an integration time of the region of interest such that the resulting frame rate is greater than the frame rate that would be realized if an area of the imaging sensor larger than the region of interest were read out.

In some embodiments, processing the image data further involves centering the image data such that a region of interest is substantially centered when the image data is displayed on the monitor. In some embodiments, centering the image data involves: retrieving a set of centering data from a nonvolatile memory module located in the camera body; and adjusting a relative position of the image data within the output monitor data based on the centering data. In some embodiments, this method may further involve generating a bounding box and not displaying sections of the image data outside the bounding box.

In various embodiments, processing the image data may involve gamma correcting the image data, denoising the image data, filtering the image data, depixelizating the image data, white balancing the image data, and/or formatting the image data for display to a display device. Optionally, the method may further involve, before advancing the elongate sheath into the human or animal subject, inserting the sheath into a lumen of a medical device, where the sheath is advanced into the subject by advancing the medical device into the subject. In some embodiments, the medical device is configured for use in a ureter of the human or animal subject, and the advancing step involves advancing the medical with the inserted sheath into the ureter. In some embodiments, the medical device comprises a camera system. In some embodiments, inserting the sheath comprises mating a mating feature on the camera body with a corresponding mating feature on a proximal end of the medical device. The method may optionally further include: removably coupling the camera body with the medical device via locking features on the mating feature and the corresponding mating feature; and identifying the medical device with a processor in the camera body.

In another aspect, a medical fiber optic camera configured for use in a ureter of a human or animal subject may include: an elongate sheath having a proximal end, a distal end, and an outer diameter of no more than approximately 0.7 millimeters; one or more illumination optical fibers disposed within the sheath; an imaging bundle disposed within the sheath and comprising at least one fiber optic clad and multiple fiber optic cores; a mechanical structure fixedly attached to the proximal end of the elongate sheath; and a mating feature on the mechanical structure for facilitating coupling of the camera with a medical device, where the sheath is configured to fit within a lumen of the medical device.

In one embodiment, the one or more illumination optical fibers comprise about 20 to about 40 illumination fibers. In various embodiments, the medical device may be, but is not limited to, a urinary stone removal catheter device, a guide catheter, other catheter devices, a steerable sheath, an endoscope, or an access sheath.

In another aspect, a method of imaging a ureter of a human or animal subject may involve: inserting an elongate sheath of a fiber optic camera, containing one or more illumination optical fibers and an imaging fiber bundle, into a lumen of a medical device configured for use in a ureter, wherein the sheath has an outer diameter of no more than approximately 0.7 millimeters; mating a mating feature of a mechanical structure of the fiber optic camera coupled with proximal ends of the one or more illumination optical fibers and an imaging fiber bundle with a corresponding mating feature of the medical device; advancing the medical device into the ureter with the sheath residing in the lumen of the device; illuminating the ureter with the one or more illumination optical fibers; and transmitting light information through the imaging fiber bundle toward the mechanical structure of the camera.

In some embodiments, the method may also include converting the transmitted light information into image data; and transmitting the image data to a video processing console or a video display monitor. In various embodiments, the medical device may be a urinary stone removal catheter device, a guide catheter, other catheter devices, a steerable sheath, an endoscope, or an access sheath. The method may also further include removably coupling the camera body with the medical device via locking features on the mating feature and the corresponding mating feature. The method may also include identifying the medical device with electronic circuitry in the mechanical structure.

These and other aspects and embodiments are described in greater detail below, in relation to the attached drawing figures.

### BRIEF DESCRIPTION OF DRAWINGS

FIG. 1 is a diagrammatic representation of a medical imaging system, according to one embodiment;

FIG. 2 is diagrammatic representation of an electronic subsystem of the imaging system of FIG. 1;

FIGS. 3A and 3B are frontal views of a console and monitor, illustrating a method for adjusting a position of an image on the console and monitor, according to one embodiment;

FIGS. 4A and 4B are end-on and side views, respectively, of a portion of the imaging system of FIG. 1, including a fiber bundle and an imaging bundle ferrule;

FIGS. 5A and 5B are side and cross-sectional views, respectively, of a camera and housing, according to one embodiment;

FIGS. 6A and 6B are perspective views of two different embodiments of fiber optic cameras being inserted into a medical device;

FIG. 7 is a flow diagram, illustrating a method of processing images using an imaging system as described herein, according to one embodiment; and

FIG. 8 is a flow diagram, illustrating a method of using a disclosed embodiment of an integrated medical imaging system.

### DETAILED DESCRIPTION

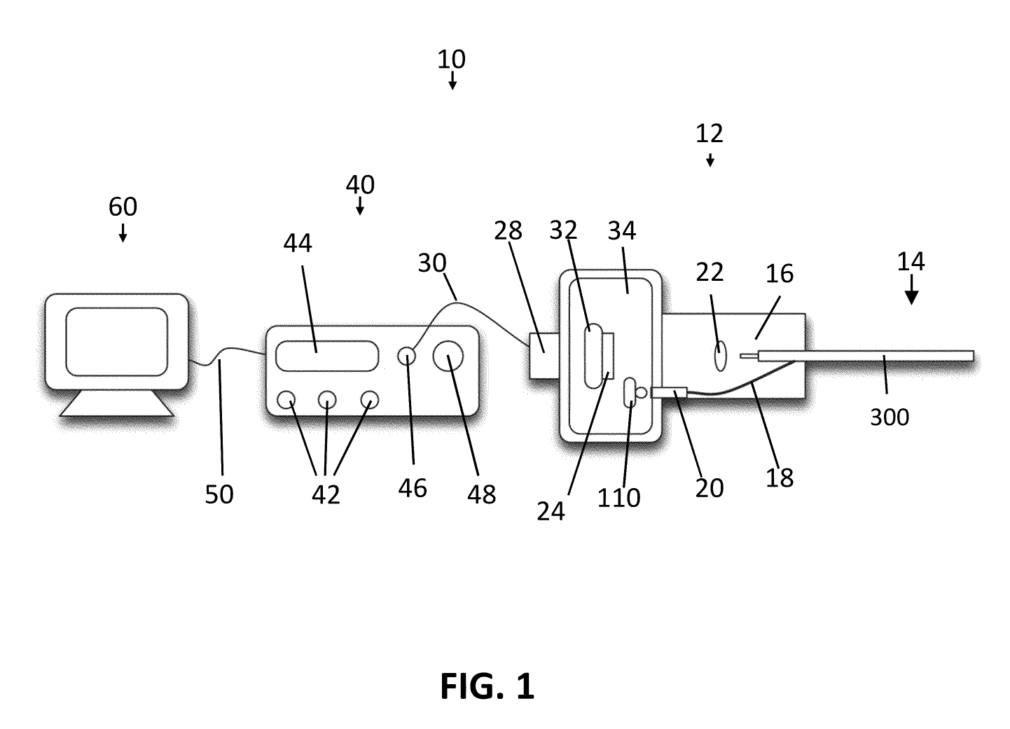

Referring to FIG. 1, in one embodiment, a medical imaging system 10 may include a fiber optic camera 12, a video processing console 40 and a display monitor 60. In alternative embodiments, system 10 may include only camera 12 and video processing console 40 or only camera 12. However, for ease of description, monitor 60 and video processing console 40 are described as part of system 10 in this embodiment. (Neither FIG. 1 nor any subsequent figures are drawn to scale. Various devices and parts of devices in various figures may be magnified, relative to other devices and parts, to enhance clarity of the figures.)

Fiber optic camera 12 may include a fiber bundle 14, which includes an outer sheath 300 (or “bundle sheath”) that houses a fiber optic imaging bundle 16 and multiple fiber optic illumination fibers 18. Sheath 300 also typically houses a lens at or near its distal end (not visible in FIG. 1). Fiber bundle 14 is fixedly attached to a camera body 36 (or “mechanical housing” or “handle”), which houses a number of components of camera 12. For example, camera body 36 may include one or more additional lenses 22 and imaging sensor 24. Imaging bundle 16 may collect light from the location being visualized by camera 12, and illumination fibers 18 may transmit light to illuminate the scene. Light information from imaging bundle 16 passes through at least one lens 22 for focusing and/or magnification, before arriving at imaging sensor 24. Imaging sensor 24 generates electrical signals, which represent image data. Imaging sensor 24 may be mounted on a printed circuit board (PCB) 32 with circuits to facilitate power and control of imaging sensor 24 and other electronic peripherals. Illumination fibers 18, or portions thereof, may be bundled into a ferrule 20, which may be optically coupled to an illumination source. The illumination source may be, for example, a light emitting diode (LED) 110; however, other illumination sources may be used in alternative embodiments. Camera 12 may further include a connector 28, which is electrically coupled with PCB 32, and a cable 30, which connects connector 28 with video processing console 40. Connector 28 may be directly electrically coupled to LED 100 or indirectly electrically coupled to LED 100 through PCB 32. A number of these features of camera 12 will be described in greater detail below.

The term “integrated,” as used herein, generally refers to some embodiments of camera 12, in which one or more of LED 110 (or other light source), imaging sensor 24 and electronics subsystem 34 are housed within camera body 36, which is fixedly (or “permanently”) attached to fiber bundle 14. In other words, these features are all included in one unit. This integrated configuration of camera 12 has certain advantages, such as that there is no need for an external, separate illumination source. This and other advantages are described in more detail in this disclosure. In alternative embodiments, fiber bundle 14 may be removably attached to camera body 36, and this removability may have alternative advantages. The term “integrated” may thus also refer to a subset of integrated features, such as LED 110, imaging sensor 24 and/or electronics subsystem 34 being integrated into camera body 36. Other alternative embodiments might not include integration of components as described herein. For example, some embodiments may include fiber bundle 14 coupled with a mating member (or “mating feature”) for coupling with a corresponding mating feature on a medical device, such as a camera, catheter or other device. Therefore, while some embodiments are described herein as being integrated or “fully integrated,” alternative embodiments may be partially integrated or not integrated.

In some embodiments, LED 110 may generate a significant amount of heat during use of camera 12, depending on the drive level used in the system. To that end, it may be advantageous to thermally couple LED 110 to camera body 36, so that camera body 36 acts as a heat sink or heat dissipation device. In some embodiments, for example, LED 110 may be mounted to a metal-clad PCB, which is then fixed to camera body 36. Thermal pastes, thermal adhesives, thermal materials, and other thermal conductors maybe used to more efficiently thermally couple LED 110 to camera body 36 by creating, for example, a thermal bridge. This thermal coupling uses camera body 36 as a heat sink for the heat generated by LED 110 and allows for higher drive currents without overheating electronics in subsystem 34 and without overheating camera body 36. In the case of handheld applications, this is advantageous.

Cable 30 typically has at least three conductors, but in some embodiments it may have fewer or more conductors. For example, cable 30 may include a power conductor, a ground conductor, and an image data conductor for sending image data from camera 12 to video console 40. In one embodiment, cable 30 and connectors 28 and 46 each have six conductors: two for power and ground, two for inter-chip communication (I2C), and two for low voltage differential signal (LVDS) used to transmit image data. Video may be comprise a plurality of discrete images displayed quickly enough to give a viewer an illusion of continuous image capture. The image data, therefore, may be used to generate a video output. The I2C bus may facilitate the control of myriad parameters of the electronics in camera 12. Among other parameters that may be modified include sensor 24's gain, exposure, and sensitivity, the drive level of LED 110, and other suitable parameters. Several other control buses may be used, including serial peripheral interface (SPI), 1-wire, and other control buses. Additionally, this control bus may easily be modulated over the power lines or otherwise embedded into other signals, in order to reduce cable conductor count. In one embodiment, image data and control signals may be modulated on the same conductors, resulting in a total of four conductors.

Video console 40 contains an electronics system that is electrically coupled to connector 46. The electronics system may contain a processor configured to run a combination of hardware and software video processing algorithms. Additionally, the electronics system may be configured to store and retrieve any received image data through cable 30 into a frame buffer. The electronics system of console 40 may also contain a display driver, which may be used to aid in generating an output capable of displaying an image to monitor 60. The same output could be used as an input to a video recording device, transmission device, and the like not shown for simplicity. The display driver may generate one or more outputs capable of driving any number of common or custom video buses, including VGA, DVI, HDMI, s-video, composite and other buses. Cable 50 carries the video console 40's output, which contains image data to monitor 60, which displays the resulting video. In alternative embodiments, wireless transmitters and receivers or other wireless communications may be used, in which case video cable 50 may not be required. In other alternative embodiments, video processing console 40 may include a video display monitor, so that it is not necessary to connect to a separate monitor 60.

Video processing console 40 may include optional control dials 42, power switch 48, and screen 44. Control dials 42 may provide a mechanism whereby the user modifies various properties or configuration settings of the imaging system. Screen 44 may display various status information of the imaging system (for example, current system settings, elapsed use time, and other status information). Power switch 48 may provide a convenient way to turn console 40 and/or camera 12 on and off.

In various alternative embodiments, any or all of the components and/or features of video processing console 40 described above may be included in camera 12 instead. In fact, in some embodiments, system 10 may include only camera 12, and video processing console 40 may be eliminated. In such embodiments, video processing may be performed by camera 12 or by some separate device that is not a part of system 10.

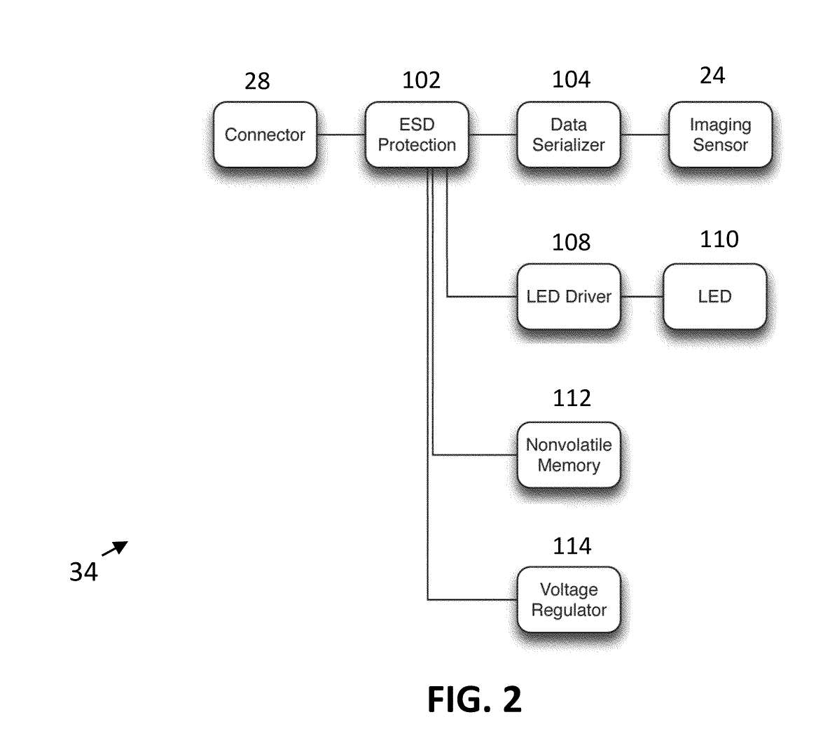

FIG. 2 shows a detailed view of electronics subsystem 34 by schematically illustrating various electronic components of subsystem 34, which may be located on one or more PCBs. Any number of PCBs may be used to implement subsystem 34. In some embodiments, multiple conductors from connector 28 may be routed through an optional electrostatic discharge (ESD) protection circuit 102, which then feeds the remaining electrical components of subsystem 34. Generally speaking, any electrical circuit requires power. Voltage regulator(s) 114 may regulate power from connector 28 to one or more nominal system voltages. In the case where more than one voltage is required in the system, regulators local to subsystem 34 may reduce the number of conductors required in connector 28 and cable 30. For example, if subsystem 34 requires more than one power supply, a single power line may be regulated to the requisite supply voltages.

FIG. 2 shows that electronics subsystem 34 includes LED 110 and LED driving circuit 108. LED 110 may be a single LED or a group of multiple LEDs. Typically, the imaging system 10 shown in FIG. 1 will use a white LED for illumination. A color temperature on the order of about 4000K to about 8000K should be sufficient for proper illumination. In some embodiments, however, any number of other wavelengths may be used for illumination. LED 110 is driven by driving circuit 108 (or “LED driver”). Since LEDs are inherently current driven devices, LED driving circuit 108 can properly regulate and maintain a desired current drive to LED 110, to realize a stable illumination level. In some cases, driving circuit 108 uses a reference resistor and current mirror to drive a desired amount of current. The drive current is a function of the value of the resistor. In one embodiment, driving circuit 108 uses a digital potentiometer instead of a fixed resistor. The digital potentiometer's value can be controlled over the control bus, allowing for illumination control.

This embedded and integrated illumination system has several advantages over traditional systems that require a light box, illumination cable, and light post. First, there is no requirement for a bulky light cable extending from camera 12. As previously mentioned, light cables are prone to damage and degradation and can be yet another breakable piece of a delicate system. Second, the disclosed embodiments are more efficient than a traditional light box. A typical light box uses on the order of 100 W of power to generate requisite illumination, whereas the system described here uses on the order of 1 W of power—two orders of magnitude less than current solutions. Finally, light boxes may break, and bulbs can be costly to replace. By contrast, the lifetime of the LED 110 used in system 10 is on the order of thousands of hours, far exceeding the lifespan of a traditional light box. This integrated illumination scheme is less expensive, more robust, more ergonomic, and more efficient than traditional endoscopic illumination schemes.

FIG. 2 shows imaging sensor 24 and optional data serializer 104. Imaging sensor 24 captures the light information relayed from imaging bundle 16. Sensor 24 may convert this light information into electrical information and output the information in any number of formats including analog video (e.g. NTSC, PAL, etc), digital video (e.g. CCIR 656, H.264, etc), and digital image data (e.g. 10 bits of pixel data, a pixel clock, horizontal synchronization signal, and vertical synchronization signal). This output of the imaging sensor may be referred to as “image data” though a video stream may comprise multiple images and, therefore, the image data can be used to realize video data. In some embodiments, imaging sensor 24 is a single integrated circuit that contains circuitry to produce image data that is passed to video processing console 40 through connector 28 and cable 30. In some embodiments, the image data can directly drive a display device such as a monitor or television without the use of video processing console 40.

Imaging sensor 24 may have a particular responsivity to light, such that the more responsive imaging sensor 24 is, the more it responds to light. Responsivity may be measured in volts per lux-second or v/lux-s at a nominal wavelength of light, often 550 nM. The output of the imaging sensor pixel is voltage, and light brightness is measured in lux. A higher responsivity means more volts per unit light time. For example, a sensor with 15 v/lux-s is more responsive than one with 4 v/lux-s; given a fixed amount of light the 15 v/lux-s sensor will be roughly 3.75-times more sensitive than the 4 v/lux-s sensor and may therefore need less time to reach a comparable exposure. Generally speaking, the frame rate of the system is inversely proportional to the exposure time of the imaging sensor. A higher exposure time results in a more exposed image and a lower frame rate. In dark lighting conditions (such as inside the body), a higher exposure may be desirable, but there may be practical constraints, such as realized frame rate. For example, if it takes 1 second of exposure to properly expose the imaging sensor, then the realized frame rate is on the order of 1 frame per second (fps). This may be impractical for use in the medical context. The typical solution to imaging dark scenes is to increase the amount of light input to the scene until proper exposure can be realized at a desired frame rate. This involves increasing the number or size of illumination fibers, the brightness of the illumination source, or the coupling efficiency between the illumination source and the distal end of the illumination fibers. These solutions, however, have disadvantages, which may render them impractical for certain applications. For example, increasing the coupling efficiency between the illumination source and distal end of the illumination fibers may be very costly. Increasing the brightness of the illumination source may generate a substantial amount of heat. It may be impractical in size-constrained applications to increase the size or number of illumination fibers. Sensor responsivities of 4V/lux-s at 550 nM or greater facilitates reduced bundle diameters by not requiring as many illumination fibers as may otherwise be needed. These fewer illumination fibers may generate less illuminating than would otherwise be required to image a scene at a desired exposure and frame rate. High responsivity may allow for properly exposed images, even if there are few illumination fibers or there is poor coupling efficiency between LED 110 and illumination bundle 18. High sensitivity may also enable LED 110 to be driven at a lower power. In one embodiment, imaging sensor may have a sensitivity of 15 v/lux-s. Other embodiments may have a sensitivity of 4.8 v/lux-s; however, higher or lower sensitivities may be used, depending on desired imaging characteristics and other factors.

In one embodiment, imaging sensor 24 produces a digital representation of the image using one or more embedded analog to digital converters. In some cases, imaging sensor 24 produces between 4 and 24 bits per pixel, horizontal and vertical synchronization signals, and a pixel clock signal. Data and control can be transferred to video processing console 40 via connector 28 and cable 30. Many commercial clip-on cameras require several conductors in cable 30—one for each bit per pixel, synchronization signal, and clock signal. This may result in thirteen conductors in the case where imaging sensors use 10-bits per pixel, two synchronization signals, and a clock signal. As more conductors are required in the cable, the system becomes heavier, bulkier, and less ergonomic. Additionally, a larger connector may increase the overall size of the camera. Furthermore, the more conductors required the more expensive the system—the cost of the cable and connectors goes up substantially with the number of conductors in the system. Finally, transferring digital signals over a long distance (a cable may be on the order of several feet) is challenging. The intrinsic impedance of a cable and environmental noise means that single-ended data may become corrupted. As a result, data serializer 104 is used in one embodiment. Data serializer 104 may also be used to reduce the number of conductors needed to transmit image data in a serialized format. For example, the data from imaging sensor 24 may be transmitted in a wide parallel format with ten signals for data and three control signals and may necessitate bulky cables to transmit the signals to various control boxes. If these signals were serialized, however, the data stream may be reduced to, for example, two serial signals rather than thirteen parallel signals. This may result in a single cable 30 having a diameter of, for example, 0.125 inches connecting camera 12 to console 40. In some embodiments, the data serializer may serialize all or only a portion of the image data. For example, if an imaging sensor outputs 24 bits of data, the serializer may only serialize the 10 most significant bits; however, other configurations are possible.

In some cases serializer 104 is a part of the imaging sensor 24 (for example, the imaging sensor integrated circuit contains a serialization stage). In other embodiments, serializer 104 is a separate circuit contained within the housing. Regardless, serializer 104 may convert the parallel pixel data, synchronization signals, and clock to a serialized data stream. Video processing console 40 contains a deserializer (not shown) to repacketize the image data. In some embodiments, this data stream is a differential data stream such as low voltage differential signaling (LVDS). Utilizing serializer 104 solves many of the aforementioned problems, since fewer conductors are required (two in the case of differential signaling), resulting in decreased cost, decreased size, and increased noise immunity. This construct is advantageous as compared to an analog video signal, since it is, for example, more noise immune.

Imaging sensor 24 may contain a variety of registers or other means of controlling settings or other operational parameters. In one embodiment, the registers may be controlled over the same control bus used by the rest of the system (for example, I2C or SPI). These settings may include gain, exposure, frame rate, image size, image position, or other settings. Video processing console 40 may have the ability to control some of these parameters.

Finally, FIG. 2 depicts optional memory module 112. This memory module may be based on an electrically erasable programmable read only memory (EEPROM), flash memory, nonvolatile memory, or the like. This subsystem has a variety of uses, which can enhance the overall imaging system. In general, module 112 serves to store a variety of parameters about camera 12. Some of these parameters may include factory calibrated or calculated parameters used by the system in FIG. 1 in order to realize a desired displayed image. For example, module 112 may contain a list of imaging sensor 24 parameters, which result in the best-realized image. Exposure, gain, frame rate, high dynamic range settings, gamma settings, white balance parameters, optical alignment, and the like may all be stored on memory module 112. Other parameters that module 112 may contain pertain to the LED 110 and LED driving circuit 108. Ideal drive current, for example, may be stored as a parameter. Data other than imaging parameters may be stored on memory module 112, for example serial number, operating parameter, version number, build date, security data, compatibility data, and other similar meta-data. These data may facilitate the system's use with different cameras 12. For example, the system in FIG. 1 may be compatible with different cameras 12, which are meant for different applications and thus have different characteristics (for example, different imaging sensors, light sources, and other characteristics). Cable 30 may operably couple memory module 112 and console 40's electronic subsystems, such that the electronic subsystems may use the information contained within memory module 112 during operation. The identifying data in module 112 may help video processing console 40 “know” which camera is connected in the system. On startup, the system may be configured to use the parameters stored in module 112 to, for example, calibrate the imaging system. This calibration may mean that the user does not need to perform one or more steps, such as white balancing the system that is typically required when using traditional endoscopic camera systems.

Other data that may be stored on module 112 pertain to usage statistics, for example the number of times the camera has been used, length of each use, and other statistics. Furthermore, a limit on the number of uses may be stored on memory module 112. Camera 12 may be meant to be used for a limited number of times (for example, disposable or “resposable” for a total of ten uses). The number of allowable uses may be stored on memory module 112, and each time camera 12 is used, the count of allowable uses may be decremented or, alternatively, an active count of uses may be stored and compared to a predetermined limit. When the use limit is reached, video processing console 40 may alert the user that the camera 12 is no longer functional. Extending this concept, console 40 may display an error message and not display image data from the camera. This may prevent the camera 12 from being used beyond its number of rated uses. The number of uses may be determined based on the number of times the camera has been connected to console 40 or a minimum elapsed time of connectivity may be used to determine a single use. This information may also allow a hospital or other medical establishment to better track the system and its use.

There are several advantages to integrating the LED 110, imaging sensor 24 and subsystem 34 in a single camera body 36, which is fixedly attached to fiber bundle 14 to provide an integrated camera 12. As previously mentioned, this embodiment of camera 12 reduces the number of cables between the endoscopic tower and handheld camera. Additionally, the illumination system is far more power efficient than traditional hundred-Watt systems. Compared to a system comprised of a clip-on camera, light box, light cable, optical eyepiece and fiber bundle, the integrated embodiments described herein contain fewer system components to maintain. This greatly reduces the burden on the medical facility to properly maintain several system components. With currently available systems, when one system component malfunctions, the facility may need to either have a backup or replace it. In the disclosed embodiments, a single component may encapsulate what may otherwise be at least five different components. If a subsystem in camera 12 fails, the entire unit is easily replaced in a single step. In one embodiment, camera 12 is disposable or “resposible” (e.g., rated for 10 uses).

There is another advantage to integrating the imaging sensor 24 into the same assembly as fiber bundle 14, rather than using a clip-on camera. The optical alignment between imaging bundle 16, proximal lenses 22, and imaging sensor 24 is a factor in realizing a proper output image. In one embodiment, the optical centers of imaging bundle 16, proximal lenses 22 and imaging sensor 24 are coaxial. Additionally, the spacing between imaging bundle 16, proximal lenses 22, and imaging sensor 24 is a factor in maintaining an in-focus image with minimal chromatic aberrations. A clip-on camera/eyepiece adds several layers of complexity, and it may be relatively easy to scratch, mar, or otherwise dirty the optical surfaces of either the eyepiece or the clip-on camera. Additionally, a clip-on camera adds two degrees of freedom in the optical path: the coaxial requirement of optical centers can shift as well as the spacing between imaging sensor 24 and the eyepiece (which effectively serves a similar purpose to proximal lenses 22). This means that excellent mechanical coupling is required between the eyepiece and camera. Any shift between the clip-on camera and the eyepiece can at best result in an image that is off center and at worst result in chromatic and other optical aberrations. The aberration in ideal spacing between the clip-on camera and eyepiece is typically fixed with an adjustment ring, which allows the clip-on camera to focus the image. Additionally, if the eyepiece or clip-on camera is damaged (for example, chipped or worn down), then it is possible that the image will be degraded. Disclosed embodiments where fiber bundle 14 and all optical elements are hermetically sealed in camera 12 do not have these issues, because, after manufacturing and inspection, it is difficult to mar or dirty the optical path internal to the camera. Additionally, during manufacturing, fiber bundle 14 (and as a result imaging bundle 16) can be adjusted to an ideal position, such that the resulting image is in the best possible focus for the system. This removes the issue of optical spacing found with the traditional approach. It further reduces the burden of focusing the system on the user. In currently available systems, the user must clip on the camera and adjust the focus. Often, during use, the focus ring is nudged or moved, accidentally moving the image in and out of focus. These user-related issues are mitigated by integrated system 10.

There remains, however, the issue of maintaining a coaxial relationship between the optical centers of all components. The coaxial relationship may be a factor in image quality (e g minimizing chromatic aberrations and maintaining proper optical apertures) and for realizing a centered image. If the light cast on imaging sensor 24 is not centered on imaging sensor 24 than the resulting image data may result in an image that is not centered. In some systems, there may be, for example, three lenses and multiple optical apertures, resulting in, for example, seven optical surfaces whose optical centers are coaxial to each other (proximal face of imaging fiber, three lenses, two apertures, and imaging sensor). The design of the camera body 36 is a factor in maintaining this relationship. Tight tolerances can ensure the spacing and alignment of lenses 22 and apertures. The alignment of imaging sensor 24 and imaging fiber bundle 16 to the system is, however, not easily solved by tight tolerances in the mechanical design of camera 12. In some embodiments, imaging sensor 24 is mechanically coupled to camera 12 by screwing or otherwise mating PCB 32 to camera body/mechanical housing 36. This may introduce mechanical slack, caused by, for example, the tolerance of soldering imaging sensor 24 to its pads on PCB 32, the pad placement on PCB 32, the mounting hole tolerance of PCB 32 and other factors. Bringing fiber bundle 14 into the proper location relative to lenses 22 focuses the system. To maintain optical alignment, camera body 36 has a channel sized for imaging bundle 16 or in some cases a ferrule. In order to slide the imaging bundle 16 in or out of the channel a sliding fit may be provided. The spacing of the sliding fit—even just a few thousandths of an inch—can be enough to degrade the optical alignment of the system. Additionally, ensuring the proper relative spacing between the proximal surface of the imaging fiber and the next optical surface in the system can be challenging. Most fiber manufactures struggle to center and position the fiber by manually rotating and moving the fiber until the image is centered, a laborious and time intensive task. Once a centered and in-focus image is realized, any movement of any optical element may result in a degraded image. If the imaging sensor needs to be replaced, for example, then the image will likely be off center on the replaced imaging sensor due to tolerance issues. Manually positioning, rotating, and adjusting components of the system until a centered, focused image is realized is the traditional solution but presents a number of challenges. The embodiment of system 10 shown in FIG. 1 can realize optical centering of the image without many of the traditional challenges by taking advantage of memory module 112 and video processing console 40.

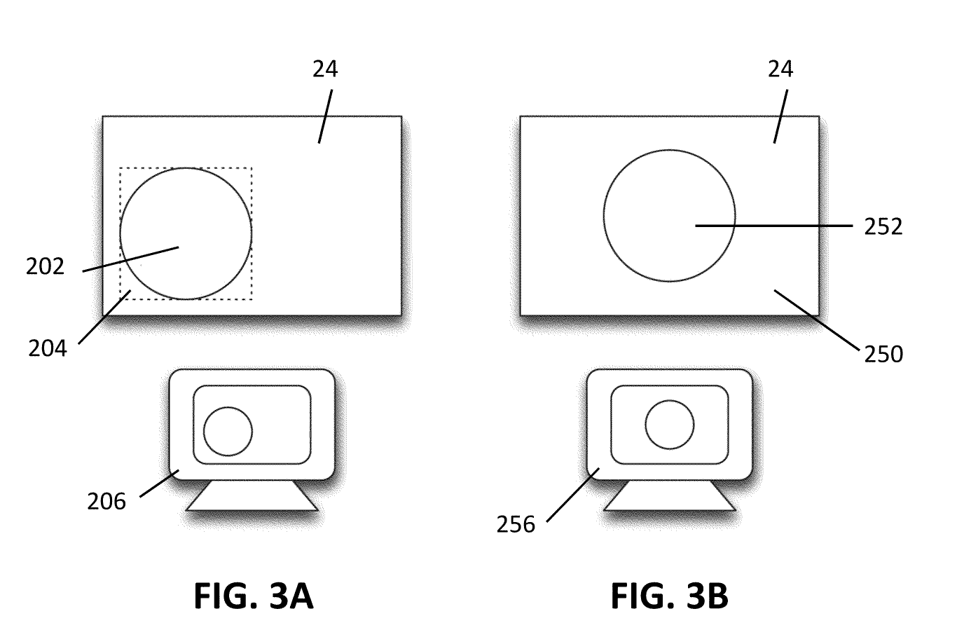

Referring now to FIGS. 3A and 3B, one solution to centering the image is performing image detection, identifying the center of the image cast by imaging fiber 16, and compensating by shifting the image in software prior to displaying the image readout to monitor 60. Due to the integrated nature of some of the disclosed embodiments, there is an alternative and potentially superior solution, which takes advantage of memory module 112. During the manufacturing process, lenses 22 are installed in camera body 36, and imaging sensor 24 is mechanically coupled to camera body 36. In some embodiments, this may be accomplished with four mounting screws. The optical alignment between sensor 24s′ optical center and the lenses' optical center may be off by several pixels. FIGS. 3A and 3B show schematic representations of imaging sensor 24 with image 202 or image 252 cast by imaging bundle 16 and lenses 22. In FIG. 3A, image 202 is off-center. Centered image 252, shown in FIG. 3B, is the desired scenario. Imaging bundle 16 is approximately optically centered over lenses 22 via a tight sliding fit. Typical optical tolerances are on the order of a few thousandths of an inch, for which camera body 36 may accommodate. Fiber bundle 14 is moved in and out until an in-focus image is realized. The image may be off-center due to the aforementioned mechanical tolerances of mating sensor 24 to the camera body and aligning the fiber 16 with lenses 22. The traditional solution is to rotate and reposition the fiber until a centered image is realized. By contrast, there are at least two simple approaches to centering the image using the disclosed embodiments. The first approach is to read the entire imaging sensor's pixel array. The data from the array may be stored in memory (e.g. a frame buffer) in console 40. When reading out the image to monitor 60, which may have a resolution greater than a region of interest of the pixel array, a region of interest of the pixel array may be padded by arbitrary data (for example a background color) to generate an image with a resolution equal to the monitor image with the region of interest substantially centered in said image. This effectively crops out sections of the pixel array and replaces said sections with padded data used to fill the remaining pixels in the monitor image. The coordinates of the region of interest relative to sensor 24 array may be stored in nonvolatile memory module 112 and read by console 40. The coordinates may be stored in various ways. The data stored on nonvolatile memory module 112, which represents the coordinates of the region of interest, may be referred to as “positioning data.” For example, the coordinates of a bounding box 204, in FIG. 3A, may be stored. Bounding box 204 may be used to ignore or not display sections of the imaging sensor output data or data that does not contain image data of interest (for example, the portions of the video signal that are not exposed by the imaging fibers).

Alternatively, the center coordinate of image 202 cast by fiber 16 may be stored, along with a radius in pixels of the image. Alternatively or in addition, data relating to the upper left and lower right coordinates may be stored. Using this data, console 40 may adjust the relative position of the output image on the monitor. FIG. 3A shows monitor 206 with the original off center image 202, and FIG. 3B shows monitor 256 after console 40 uses region of interest information to adjust the relative position of output image 252.

Alternatively, the parameters of sensor 24 may be modified to read out a particular region of interest directly from sensor 24. Sensor 24 may have adjustable parameters, including the readout start row, column, and readout image size. By adjusting these parameters, a region of interest can be read from sensor 24. The ideal start/stop row/column may be stored in module 112 and read by console 40. Console 40 may then write these parameters to imaging sensor 24 and as a result read an image with the desired region of interest directly from sensor 24.

The above-described approach may provide several advantages. Camera 12 in FIG. 1 and similar imaging systems may be designed to have a fill factor less than 100%. For example, the image cast by fiber 16 and lenses 22 may have a maximum dimension that is less than the smallest dimension of the imaging sensor 24. In other words, the image cast by fiber 16 and lenses 22 may not expose a portion of the imaging sensor. This is by design for a few reasons, including the fact that a 100% fill factor may result in undesired pixilation effects of the fibers in the imaging bundle. Additionally a 100% fill factor may result in more complicated or expensive proximal lenses 22. Finally, an image cast by fiber 16 and lenses 22 that is equal to the smallest dimension of the imaging sensor 24 requires perfect optical alignment to capture the entire image. Any shift in the optical alignment will result in part of the image case by fiber 16 and lenses 22 to “fall off” the imaging sensor 24. A fill factor of less than 100% means that the image cast on sensor 24 is necessarily smaller than sensor 24. Reading out the ROI directly from sensor 24 means, therefore, that not all pixels of sensor 24 are read. The frame rate of sensor 24 is a function of the integration time of sensor 24 and the readout time of sensor 24. In the worst case scenario, there is no overlap between the integration and readout, such that frame rate is roughly approximated as the inverse of the sum of integration time and readout time. In many cases, however, there is overlap between the two, such that the frame rate is faster than this worst case. Regardless, the number of pixels read from sensor 24 directly influences frame rate. For a fixed pixel clock, the more pixels read the lower the frame rate. By reading a smaller region of interest, the number of pixels read from sensor 24 decreases, which means that the frame rate can increase “for free,” as compared to reading the entire imaging sensor. Alternatively, the frame rate can be held constant and the integration time increased “for free,” resulting in greater sensor exposure. The latter may be useful in lower light scenarios. Some balance between increased frame rate and exposure may also be realized. Disclosed embodiments may produce useful imaging at a frame rate of about 30 frames per second to about 60 frames per second; however, some configurations of disclosed embodiments may be operable at even higher frame rates.

Even it were possible to properly align all optical components via tight tolerances of camera 12's mechanical structure, the cost of realizing such a configuration may be unnecessarily high. The solutions presented above offer a simple and low cost technique to center the resulting image. These techniques may not be possible in systems that are not fully integrated. As a result storing centering or positioning data/parameters on nonvolatile memory module 112 is advantageous.

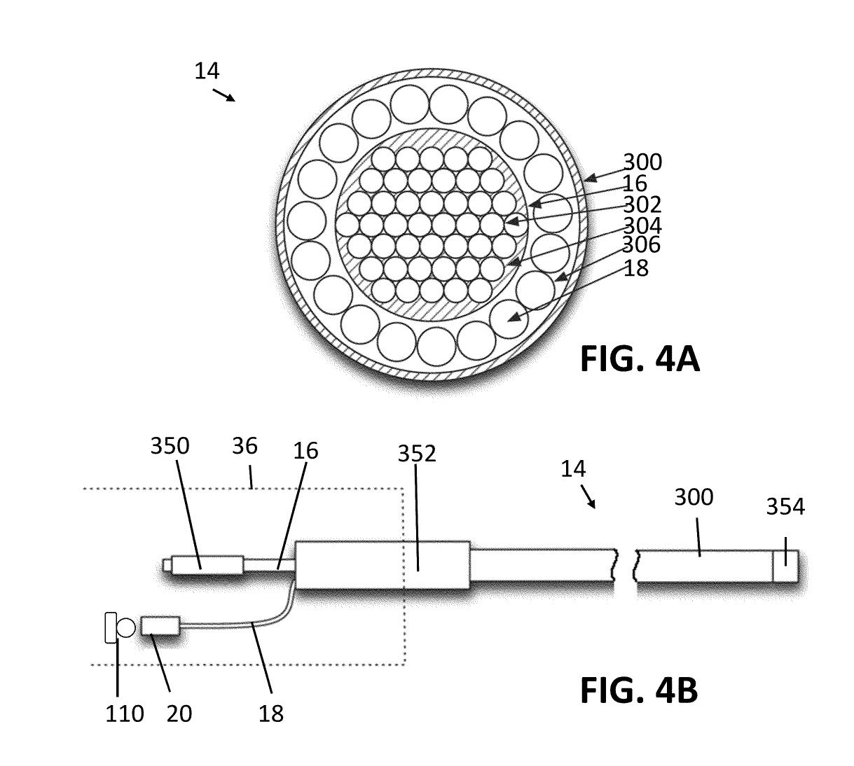

FIGS. 4A and 4B show fiber bundle 14 in greater detail. FIG. 4A shows a cross section of fiber bundle 14, while FIG. 4B shows a side view of fiber bundle 14. FIG. 4A shows fiber bundle 14 comprising outer bundle sheath 300, imaging bundle 16, and illumination lumen 306 comprising at least one illumination fiber 18.

As shown, imaging bundle 16 may comprise one or more fibers 302. The word “fiber,” in reference to imaging bundle 16, means at least one fiber optic core, which is surrounded by a fiber optic clad, thus resulting in a fiber optic waveguide. The spatial resolution of imaging system 10 is directly proportional to the number of fibers in imaging bundle 16 and the size of the area being imaged. Generally speaking, the more fibers in imaging bundle 16 the higher quality the resulting image. There are several viable configurations of imaging bundle 16. As shown, imaging bundle 16 may comprise one or more fibers 302, which in one embodiment are comprised of one or more fiber optic cores surrounded by fiber optic cladding 304 common to all fiber optic cores. However, other configurations of imaging bundle 16 are possible. For example, in some embodiments, fibers 302 are complete fibers with individual cores and individual cladding. In one embodiment, imaging bundle 16 may comprise on the order of about 1,000 to about 10,000 individual fibers. In preferred embodiments fibers 302 may have core diameters between 1 and 30 microns, but other sizes may be used.

Similarly, illumination fibers 18 may include various configurations of one or more fibers. Illumination fibers 18 may comprise one or more individual illumination fibers 18 comprised of an individual core and individual cladding. In another embodiment, illumination fibers 18 may comprise a single common cladding surrounding a plurality of fiber cores. Illumination fibers 18 may also be a plurality of fiber cores each with their own individual cladding. As shown in FIG. 4A, illumination fibers 18 may comprise a plurality of illumination fibers 18 surrounding imaging bundle 16. In other embodiments, there may be a plurality of illumination fibers 18 adjacent to, separate from, or otherwise related to imaging fibers or imaging bundle 16.

In another embodiment, the fiber bundle 14 may comprise 3,000 imaging fiber cores sharing a common clad and about twenty to about twenty-five illumination fibers 18. In some embodiments, one end of illumination fibers 18 may have a total core surface area of less than about 0.00003 square inches, for example, about 0.000025 square inches. Illumination fibers 18 may be directly coupled to LED 110, which provides a white light source. In a directly coupled configuration, the illumination fibers may be separated from LED 110 by approximately 0.005 inches, but other distances are possible. One or more lenses or other optical elements may be used in order to focus the light from LED 110 into illumination fibers 18.

Illumination fibers 18 provide illumination to the scene of interest. In some embodiments, illumination fibers 18 have diameters between 25 and 100 microns. Illumination fibers 18 are housed between bundle sheath 300 and imaging bundle 16 in illumination fiber lumen 306. The number of illumination fibers 18 in fiber bundle 14 is a function of the diameter of illumination fibers 18 and the cross sectional area of illumination fiber lumen 306. A larger bundle sheath 300 or smaller imaging bundle 16 may increase the size of lumen 306, allowing for more illumination fibers.

Certain applications favor certain parametric designs. Imaging gross anatomy in a large open volume may favor increasing the number and or size of illumination fibers 18. This is because imaging a large open volume necessitates illuminating the entirety of the volume. By contrast, imaging a tissue surface from a very short distance may favor increased spatial resolution. Typically, the constraining metric is the outer diameter of fiber bundle 14, which is the outer diameter of bundle sheath 300. In some embodiments, the outer diameter of fiber bundle 14 is between approximately 0.25 mm and approximately 1 mm. In more specific embodiments, fiber bundle 14 may have an outer diameter of no more than approximately 0.7 mm, or more preferably no more than approximately 0.6 mm. In one embodiment, imaging bundle 16 has an outer diameter between about 200 microns and about 550 microns and a total length of between 15 cm and 200 cm. In some embodiments, the wall thickness of bundle sheath 300 is between about 0.025 mm and about 0.127 mm, with the remaining space in lumen 306 to be maximally packed with illumination bundle 18.

FIG. 4B shows a side view of fiber bundle 14. An objective lens (not shown) may be optically coupled to the distal end of imaging bundle 16 and may be configured to collect light from the location being visualized by camera 12 and carry it down the length of the fiber. The objective lens may be a gradient index (GRIN) lens or single-element or multi-element construction. In some instances, the lens(es) may be molded, ground, or otherwise fabricated. An optional lens sheath may help protect the delicate optics. An optional lens sheath (not shown for simplicity) may help protect the delicate optics. The lens sheath may further help join and optically center imaging bundle 16 and objective lens.

An optional distal optical sheath 354 may encase the distal contents of fiber bundle 14 and help protect the distal optics. Distal optical sheath 354 may be constructed of stainless steel or other biologically inert materials. Distal optical sheath 354 may further protect the connection between the objective lens and imaging fiber 16. In one embodiment, the distal tip of distal optical sheath 354 is roughly flush with the distal optical surface of the objective lens and the distal end(s) of illumination fiber(s) 18. In the same embodiment, the proximal end of distal optical sheath 354 is more proximal than the joint between imaging bundle 16 and the objective lens. In some embodiments the overall length of distal optical sheath 354 is roughly 0.2 inches.

Bundle sheath 300 (also referred to herein as “outer sheath 300”) provides mechanical strength to the overall assembly, may protect delicate fibers, and may be configured to help reduce friction when fiber bundle 14 is pushed or inserted into a catheter or other lumen. Bundle sheath 300 may be made of polyimide, polytetrafluoroethylene (PTFE), polyether block amide (for example, as sold under the trade name PEBAX), or any other suitable flexible material. In some embodiments, bundle sheath 300 is made of polyimide or a polyimide variant and is darkly colored, preferably black. In embodiments that do not use distal optical sheath 354, the distal end of bundle sheath 300 is approximately flush with the distal optical surface of the objective lens and the distal end(s) of illumination fiber(s) 18.

In many embodiments, it may be advantageous to be able to slide/advance/insert fiber bundle 14 of camera 12 into/through a lumen of another device. Such devices may include a urinary calculus extraction catheter, other types of catheters, a steerable sheath, a guide sheath, a ureteroscope or any other type of endoscope, for example. For minimally invasive procedures, it is often desirable to minimize the size and profile of visualization devices used during the procedure. It is therefore desirable to minimize the clearance between camera 12 (e.g., sheath 300) and its mating lumen, while also minimizing friction between camera 12 and the lumen for ease of insertion. As a result, it may be important to design camera 12 and bundle sheath 300 for maximum pushability, while maintaining required flexibility. As the length of the lumen increases, the difficulty in advancing a flexible shaft down the lumen may also increase, due to the increased friction force. Of particular concern is kinking or breaking fiber bundle 14 while advancing camera 12 into a lumen. The contact between the lumen surface and the surface of bundle sheath 300 is prone to induce a friction, which may be overcome by advancing camera 12 forward up the lumen. Due to the flexibility of fiber bundle 14, fiber bundle 14 may bend at or near the entrance of its mating lumen. As a result, selecting the proper material for sheath 300 and designing proper spacing in the mating lumen may be beneficial. In some embodiments, sheath 300 is made of braided, coiled, or otherwise reinforced flexible polymers. This reinforcement increases the stiffness of fiber bundle 14 and facilitates the advancement of camera 12 up a mating lumen. In one embodiment, sheath 300 is made of coiled black polyimide with a wall thickness of roughly 0.002 inches. A coiled reinforcement may favor advancing camera 12 up a mating lumen over a braided reinforcement due to the increase flexibility allowed by the spacing between each coil wind as compared to a braided structure. A coil may also allow for a decreased wall thickness compared to a braid due to the lack of an overlapping wire structure.

The surface contact between bundle sheath 300 and the mating lumen creates friction during camera advancement. To that end, design optimizations that lower friction between the two surfaces may be advantageous, for example lowering the coefficient of friction between the two lumens by providing a lubricious coating may prove efficacious. The inclusion of PTFE, hydrophilic coatings, other coatings or other materials on either the outside of sheath 300 and or inside of the mating lumen may be useful. There is, however, an advantage of coating sheath 300 rather than the mating lumen. PTFE coatings, for example, are often difficult to sterilize with radiation methods such as e-beam or gamma sterilization. As a result there may be adverse effects of coating the lumen of the mating device. In the case where camera 12 is “resposable” (for example, rated for a certain number of uses) it can be shipped non-sterile and sterilized by other means (for example, autoclaves, low-temperature sterilization systems such as those sold under the trademark STERRAD, sterilization services such as those provided under the trademark STERIS, and other sterilization means). These techniques do not require radiation and may be more compatible with various lubricious coatings including PTFE. Furthermore coatings generally add system costs. It may be preferable to keep the cost of the disposable mating device low and amortize the coating cost across multiple camera uses. To that end one embodiment of sheath 300 uses a black biocompatible coil reinforced polyimide PTFE composite with a wall thickness of roughly 0.002 inches. This sheath uses coils to add pushability and PTFE to reduce the friction between the fiber bundle 14 and mating lumens. Such a sheath design may greatly facilitate the advancement of camera 12 into a lumen of a ureteroscope, endoscope or other medical device.

FIG. 4B schematically illustrates camera body 36 of camera 12 as a dashed line. Fiber bundle 14 and imaging bundle 16 are typically adhered to camera body 36 via an adhesive, such as but not limited to a glue. This adhesive serves at least two purposes. First, it helps lock fiber bundle 14 into position relative to the rest of camera 12. Second, it seals the gap between fiber bundle 14 and the inside of camera 12. The joint between camera body 36 and fiber bundle 14 is a mechanical weak point. Fatigue, bending, and similar situations can cause fiber bundle 14 to break at or near the joint between fiber bundle 14 and camera body 36. FIG. 4B shows a strain relief 352, which has a larger diameter than fiber bundle 14 and helps protect fiber bundle 14 at this joint. This strain relief 352 may be staged (for example, multiple diameters of cascading strain relief) or a single diameter strain relief. Appropriate materials include braided or coiled polyimide, polyether block amide (for example, as sold under the trade name PEBAX), nylon, stainless steel, and other materials. In one embodiment, the outer diameter of strain relief 352 is roughly 0.01 inches larger than the diameter of fiber bundle 14. The length of strain relief 352 can be tailored for different applications, but generally lengths on the order of 10 mm to 40 mm are appropriate.

FIG. 4B also illustrates imaging bundle ferrule 350. Ferrule 350 may be useful in positioning imaging fiber bundle 16 within camera body 36 and provide a surface, which can be adhered or otherwise bonded to a member of camera body 36. A setscrew, for example, can be used to apply pressure and consequently affix imaging bundle ferrule 350 without exerting a potentially harmful force to imaging bundle 16 itself. FIG. 4B also illustrates the bundled illumination fibers 18 and ferrule 20. Ferrule 20 may be bonded or otherwise fixed in a desired location relative to LED 110 of FIG. 1.

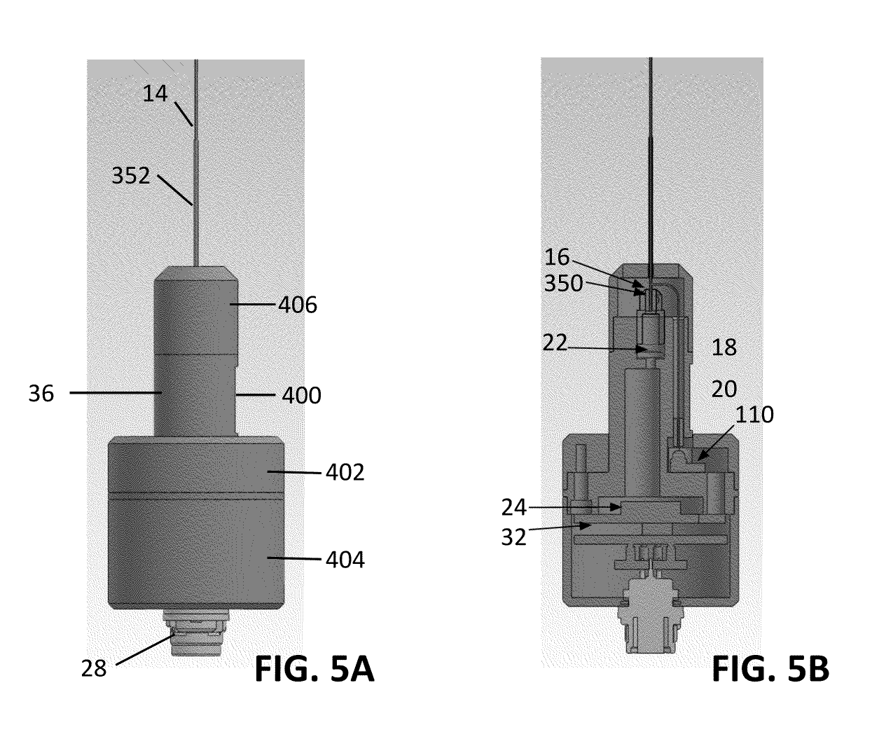

FIGS. 5A and 5B show an exemplary camera body 36 of camera 12, in two different views. FIG. 5A shows a side view of camera 12 and camera body 36, while FIG. 5B shows a cross sectional view. In one embodiment, the overall length of camera body 36 is about 0.5 inches to about 3.0 inches. In one embodiment, the widest point of camera body 36 is about 0.5 inches to about 1.5 inches. These dimensions facilitate holding of camera body 36 by a hand and result in a lightweight, easy to use, and ergonomic design. In some embodiments, camera 12 mates into other devices. Namely, fiber bundle 14 can be advanced into a mating lumen or space in another device, in order to augment said device with direct vision that may otherwise not be part of the other device. Robust mating between camera 12 and the mating device may ensure both proper location of the tip of fiber bundle 14 relative to the mating device as well as ensuring a mating connection, which will not damage camera 12 or the mating device.

Many medical devices that use separate cameras, which are advanced into said devices, rely on Tuohy Borst or other traditional off-the-shelf medical device connectors. These connectors use a silicone gasket to cinch down on the bundle of the camera. A reusable fiber optic camera may be advanced into a disposable instrument, and a Tuohy Borst adapter attached to the mating instrument may be closed tightly on the fiber optic bundle to lock the bundle's position relative to the disposable instrument. This presents a number of drawbacks. First, the Tuohy Borst adapter puts pressure on the fiber bundle. The fibers in the bundle are often very delicate; even minor forces can break the illumination fibers surrounding the imaging bundle. With enough force, the imaging fibers can also break. Furthermore, the Tuohy Borst puts a variable pressure on the bundle, depending on how hard the user tightens the connector, such that, even if there is a “safe” force that will not damage the fiber bundle, it is the user's responsibility to ensure that said force is not exceeded.

A second drawback is that Tuohy Borst adapters may cause the weight of the mechanical structure attached to the bundle to be significant relative to the weight of the bundle itself. As mentioned above, the mechanical structure attached at the proximal end of the fiber bundle could include an eyepiece, clip on camera, light cable, or portable light source; each of these has a mass that is substantial relative to the fiber bundle. As a result, mating to the bundle without supporting the weight of the back end results in a weak point directly at the point where the Tuohy Borst or other connector is attached to the fiber. If the mating device is moved, then the proximal end of the fiber optic camera could be dragged around by the mating device. This may lead to bundle damage. It is easy to imagine the backend of the fiber bundle falling off a table, getting snagged on another object, or other situations that may induce substantial stress in the fiber bundle. In some cases, the bundle might move relative to the mating instrument, which may have adverse clinical effects. In other cases, the bundle may simply break mid-procedure.

A better solution is to mate the camera body—for example, housing 36 or a mechanical housing—to another device using a mating feature 400, and thus lock the position of the bundle tip to the mating device. One embodiment of mating feature 400 may be a flat portion of housing 36, which in some embodiments is used to mate another device to camera 12. In alternative embodiments, a radially asymmetric feature may be substituted for mating feature 400. In some embodiments, the mating device may use a setscrew, cam, lever, latch, or spring to press on mating feature 400, thus constraining camera 12 in the handle or other portion of the mating device. Alternatively, mating feature 400 may comprise an external thread on a portion of housing 36 that may be used to screw in camera 12 into a mating device. Other latching mechanisms, such as a spring-loaded pin or ring, may be used to secure camera body 36 onto mating feature 400.

Compared to solutions where there are discrete adjustment steps (for example, discrete locations where camera 12 can be locked into place relative to a mating device), both the above-described solutions have the advantage that they are “infinitely adjustable”. In other words, it is easy to achieve small adjustments in the relative positioning of camera 12 and a mating device. In the case of mating feature 400, the locking device (for example, a setscrew, cam, or other locking device) can lock anywhere along the flat surface, allowing for small adjustments. In the case of the external thread, camera 12 can be screwed inwards until a desired relative positioning is found. Small adjustments may be necessary to account for tolerance issues in manufacturing and assembly. For example, the locking features may allow a connection between the mating feature and the corresponding mating feature to be slidably adjusted to ensure alignment within approximately 0.5 mm. In another embodiment, the location of the distal tip of the camera and the distal tip of the device can be slidably adjusted to ensure alignment within approximately 0.5 mm.

Mating feature 400 has another advantage over the Tuohy Borst and other fiber mating systems, in that mating feature 400 may orient the fiber relative to the mating device when the mating device mates to the bundle. This is important in applications where the user needs to navigate the medical device to a desired location by vision. Without proper orientation, there is no intuitive correlation between the user's hand movements (for example, left, right, up, or down) and the “motion” of the resulting video, such that the user may identify an object of interest in the left half of the image and navigate towards it by intuitively moving the device towards the left. However, without proper orientation, it is possible that moving the device to the left may guide the user to the right side of the image. Mating feature 400 may be used to ensure that camera 12 cannot rotate relative to the mating device by providing only one way to insert camera 12 into the mating device and lock the two together. By design, mating feature 400 can be oriented so that it is parallel to an arbitrary and known side of the imaging sensor 24 (for example, parallel to the top side of imaging sensor 24). The mating feature (for example, a setscrew, cam, or other locking device) on the mating device can be designed with this in mind, such that the top of the imaging sensor (the top of the resulting image) is aligned with the top of the device. This may ensure that up is up, down is down, left is left, and right is right, unlike some Tuohy Borst designs where there may be some ambiguity. Mating feature 400 may also be to mate with a compatible device to ensure a useful profile and weight distribution, among other useful features. These features can be designed with particular use cases in mind, such as single handed device operation.

The mating process need not be limited to mechanically mating camera 12 to another device. Mating may also include electronically mating the two devices. This may be accomplished via exposed contacts, plugs, wires, wireless pairing, and other means for operably coupling the two devices. Electronic mating may facilitate the transfer of information between the devices such as image data, alignment data, safety data, patient data, procedure data, control data, focus data, and other useful data sets. This mating may also include a validation check to ensure compatibility between system 10 and the device. If the devices are not compatible, then one or more of the devices may alert the user, cease functioning, operate at a different level or at a different configuration, or combinations thereof.

Another advantage of mating camera body 36 to another (“mating”) device is related to thermal dissipation. LED 110 can produce a substantial amount of heat. If designed correctly, the mating device may shield any or all portions of camera body 36, which may act as a heat sink for LED 110. This may result in a better user experience and not expose the user to any warm or hot surfaces. Mating other devices to camera body 36 allows the mating of a reusable or “resposable” camera with a disposable instrument.

FIG. 5A shows other design features, such as LED cover 402 and back cap 404. These pieces help seal the inside of camera body 36. LED cover 402 also shields any excess light from LED 110 from escaping into the user's environment. Front cap 406 is used to seal the front end of camera 12 from the surrounding environment and, the distal end of front cap 406 may provide a flat surface that may help mating with other devices. In particular, if the mating device uses levers or the like to move internal lumens relative to camera 12 then the flat surface on front cap 406 can help “zero” a lever relative to camera 12. The lever may be designed to bottom out on the distal end of front cap 406 to allow consistent alignment of the various lumens and cameras.

FIG. 5B is a cross-sectional view of the portion of camera 12 illustrated in FIG. 5A, showing some of the components housed in camera body 36 that are described above. The mechanical components shown in FIGS. 5A and 5B can be made of machined aluminum, injection molded plastic, injection molded metals, and the like. The various mechanical components shown should be interpreted as exemplary only. Other designs are possible and in some cases preferred. In one embodiment, camera body 36 is constructed of two injection molded pieces in a clam shell configuration.

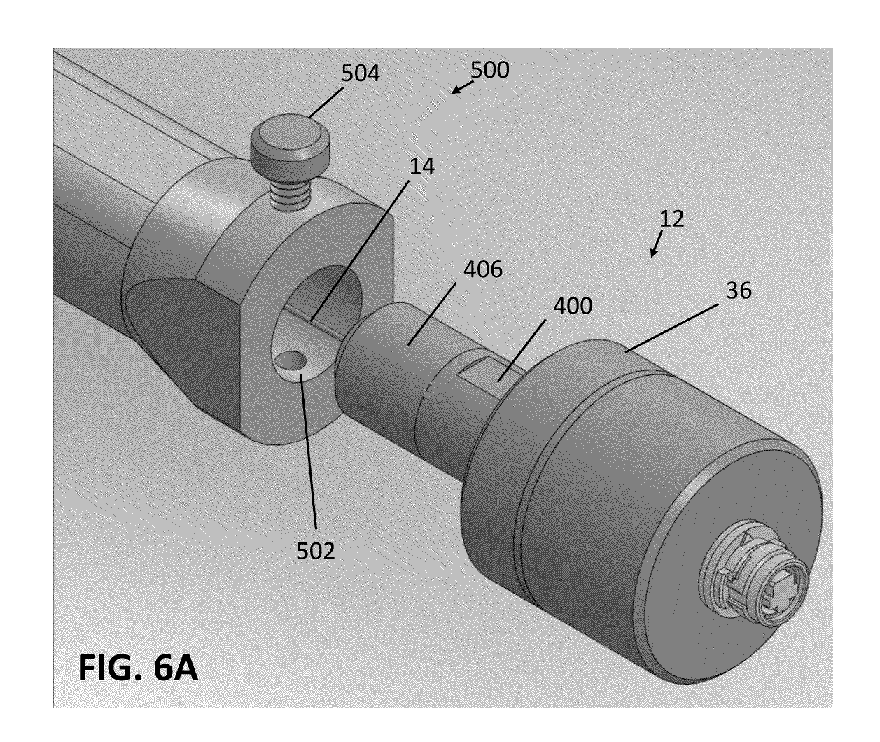

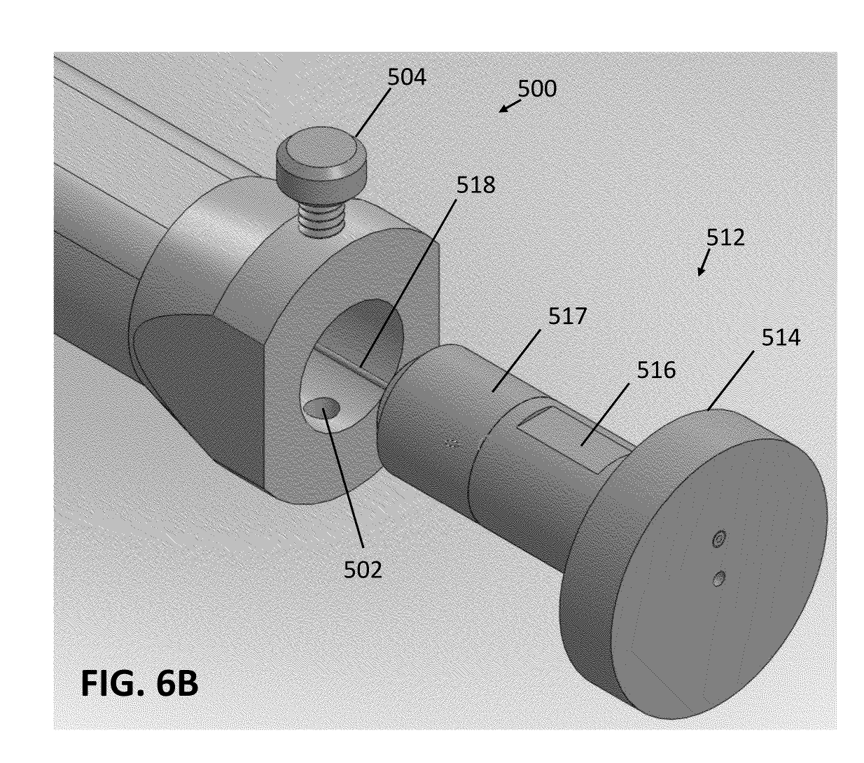

Referring now to FIGS. 6A and 6B, two alternative embodiments of cameras being inserted into a medical device 500 are illustrated. With reference to FIG. 6A and as described above, integrated camera 12 includes camera body 36 and fiber bundle 14, and the front portion of camera body 36 includes mating feature 400 and front cap 406. This front portion of camera body 36 may be inserted into a proximal opening 502 (or “lumen”) of medical device 500, which may be a ureteral stone removal catheter in one embodiment or alternatively may be any other suitable medical device, such as but not limited to those listed above. Once the front portion is inserted, a set screw 504 of medical device 500 may be tightened to contact and secure upon mating feature 400.

Referring now to FIG. 6B, as mentioned above, some embodiments of a camera 512 may not be fully integrated—e.g., may not include an internal illumination source, sensor, etc. One embodiment of such a camera 512 is illustrated in FIG. 6B. Camera 512, in this embodiment, may include a proximal mechanical structure 514 with a mating feature 516 and a front cap 517, as well as a fiber bundle 518 fixedly attached to mechanical structure 514. As with the previously described embodiment, the front portion of mechanical structure 514 may be inserted into proximal opening 502 of medical device 500, and set screw 504 may be tightened to secure camera 512 to medical device 502. Again, any suitable medical device may be mated with camera 512, according to various alternative embodiments.

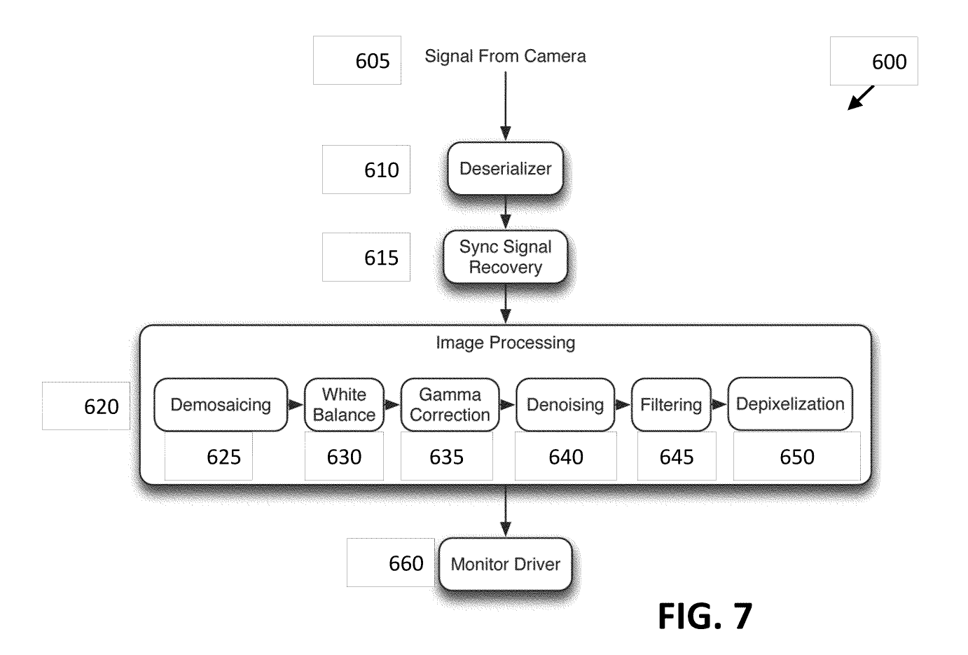

FIG. 7 is a flow diagram, illustrating a method 600 for processing images using video processing console 40, according to one embodiment. First, a signal containing image data from camera 12 is received 605 by console 40, for example via cable 30. In some embodiments, where data is serialized in camera 12, then a deserializer may be used to deserialize the data 610. In some cases, an optional synchronization signal recovery step 615 may be performed. This may be necessary if the data serialization stage embedded synchronization signal information into the serialized data stream. At this point in the method, the image data may be output to a monitor driver 660 optionally through a frame buffer or may optionally be enhanced, processed, formatted, or otherwise modified in an optional image processing pipeline 620. Monitor driver 660 may output a video bus (e.g. VGA, HDMI, DVI, s-video etc.) capable of driving a display monitor.

Image processing pipeline 620 may include all or a subset of the steps illustrated in FIG. 7. Furthermore, the order of operations within the image processing pipeline 620 is exemplary and should not be interpreted as limiting. The first illustrated step in pipeline 620 is a demosaicing step 625, which may be used in an embodiment where imaging sensor 24 utilizes a color filter array, but does not perform demosaicing. The output of the demosaicing step 625 may yield a multichannel image, which may be output to a monitor or enhanced, processed, or otherwise modified in additional image processing steps. Additional, optional image processing steps include white balancing 630, gamma correction 635, denoising 640, filtering 645 and depixelization 650. The white balancing step 630 may be used to adjust the white point of the image. Gamma correction 635 may provide a nonlinear transform to one or more of the image channels. Denoising 640 may facilitate noise reduction in the image. Filtering 645 may include the removal, attenuation, and or amplification of particular components within the resulting image. Finally, depixelization 650 may facilitate a reduction in the appearance of image pixelization due to spatial sampling associated with fiber optic imaging.

All of the above functions shown in FIG. 7 may be implemented in hardware, software, firmware, or any suitable combination of hardware, software, or firmware. The blocks shown in FIG. 7 may be implemented using programmable logic, such as an field programmable gate array (FPGA), microprocessor, digital signal processor, application specific integrated circuit (ASIC), or a combination of the aforementioned. For example, the deserializer and monitor driver may be implemented as discrete ASIC(s), while the remaining blocks in FIG. 7 may be implemented in an FPGA.

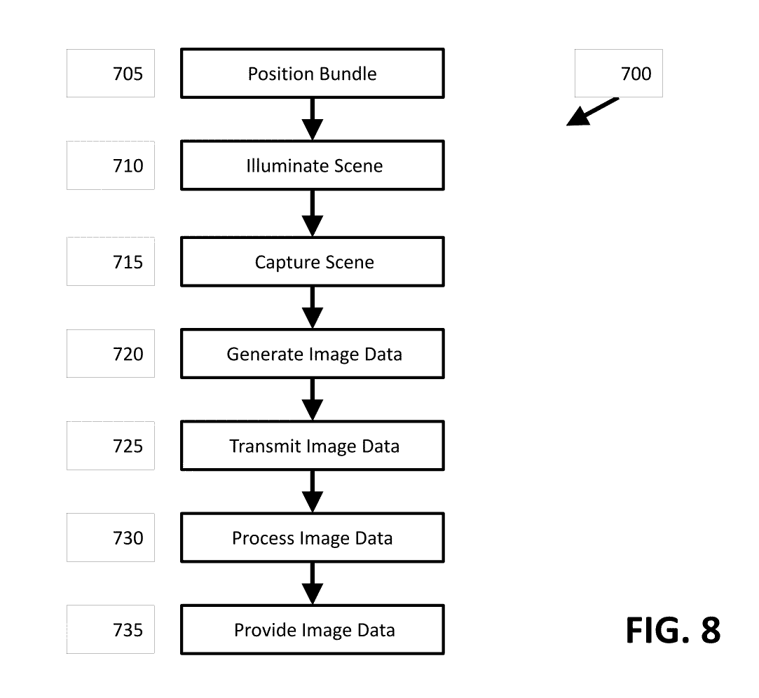

FIG. 8 shows an example method 700 of using medical imaging system 10. While step 705 is the first listed step, preliminary steps may occur beforehand. Such steps may include one or more of the following, in any order or combination: removing components of medical imaging system 10 from sterile packaging, sterilizing one or more components, connecting camera 12 and console 40 via only one cable 30, connecting monitor 60 and console 40, initializing electrical components of medical imaging system 10, comparing a camera usage statistic to a predetermined threshold, alerting a user if a camera usage statistic exceeds a predetermined threshold, setting initial illumination parameters, setting initial imaging parameters, establishing operable connections between components of medical imaging system 10, placing fiber bundle 14 in a medical device, placing fiber bundle 14 in a lumen, mating a component of system 10 with a medical device, lubricating fiber bundle 14, and other preliminary steps.

Step 705 may include advancing fiber bundle 14 into a human or animal subject to position a distal end of the fiber bundle 14 near a scene of interest in the human or animal subject. Advancing the fiber bundle 14 may include advancing the fiber bundle 14 through a medical device. The medical device may have its own camera system and step 705 may include advancing the fiber bundle 14 out of an existing camera system (for example, a ureteroscope, an endoscope, and other such devices). This configuration may allow for the medical device to a have a camera having a first set of features and the medical imaging system 10 to have a similar, different, or otherwise complimentary set of features. As an example, a smaller imaging system may be advanced out of a larger system to access tight anatomical areas.

Step 710 may include illuminating the scene of interest with illumination fiber(s) 18 of the fiber bundle 14. In one embodiment, this may be accomplished by causing light from LED 110 to travel from the proximal to distal ends of illumination bundle 18 by, for example, having the proximal ends of the illumination fibers 18 optically coupled with an LED 110 in housing 36 attached to a proximal end of the fiber bundle 14. Before, during, or after this step, there may be an additionally be the step of configuring an illumination parameter via console 40. This parameter may be the brightness, color, frequency, LED drive current, or other parameter relating to the creation of illumination.

Step 715 involves capturing light information with imaging sensor 24 in the camera body 36. Imaging sensor 24 is optically coupled with imaging bundle 16 in such a way that the imaging bundle 16 causes light to travel from the bundle's distal to proximal end and into the imaging sensor. Before, during, or after this step, there may additionally be the step of configuring a parameter of the imaging sensor 24 via console 40. The parameter may include gain, exposure, frame rate, image size, image position, sensor sensitivity, and other imaging parameters. In some embodiments, the parameter is automatically configured based on console 40, camera 12, or another device reading and acting on information stored within console 40, camera 12, or other source, for example camera use data stored on non-volatile memory.

Step 720 includes converting the light information into image data. Image data may be described broadly as analog or digital data, information, or signals relating to visual images. This step may be accomplished on the imaging sensor 24 alone or via processing light information on a combination of other sensors, processors, or microchips operably coupled to imaging sensor 24. This step may also include converting only light information captured on a particular portion of imaging sensor 24 into image data, wherein the particular portion has a surface area smaller than the surface area of imaging sensor 24.

Step 725 includes transmitting the image data from camera 12 to console 40. (This step is skipped altogether in embodiments that do not include a video processing console.) This may be accomplished by, for example, transmitting the image data from imaging sensor 24 to console 40 through cable 30, which operable couples the imaging sensor 24 to console 40. In some embodiments, this may be the only connection between the two devices. The image data may first be transferred from imaging sensor 24 to a buffer or other component of camera 12 before being transmitted console 40. In addition to or instead of being transmitted through cable 30, the image data may be transmitted wirelessly from a wireless component within camera 12 operably coupled to imaging sensor 24 to a wireless component operably coupled to console 40. This step may also include the step of serializing the video frame signal via a data serializer 104 within the camera body prior to transmission; and repacketizing the video frame signal via a deserializer within the console after transmission.

Step 730 includes the step of processing image data using the video processing console 40. This step generally involves preparing the image data for display output. The step of processing the image data may also comprise various steps for centering or otherwise altering video location within the displayed image. These steps may include centering the image data such that a region of interest is substantially centered or otherwise positioned in a desired location when the image data is displayed on the monitor. For example, in one embodiment, the console may output signal or data to the monitor, containing a background color, logo, other data, or a combination thereof. The signal or data may also contain the image data from the camera. The image data may be stored in a frame buffer (memory) in the console. In some embodiments, this data may be streamed into memory agnostic of output. On the output side, the start of reading the frame buffer may be timed such that the image data in memory is properly placed in the center or other desired position of the monitor frame.

Centering the image data may further or alternatively comprise the step of padding the image data with arbitrary data. Centering the image data may additionally or alternatively comprises the steps of: generating a bounding box and adjusting the relative position of the image data on the monitor. Centering the image data may comprise storing data comprising a center coordinate of the image data and a radius in pixels of the image data and adjusting the relative position of the image data based on the data. Alternatively or additionally, centering the image data may comprise storing data comprising information related a region of interest within the imaging sensor that is smaller than the imaging sensor (e.g bounding box coordinates). Processing the image data may also include: correcting the gamma of the image data, denoising the image data, filtering the image data, depixelizating the image data, white balancing the image data, and otherwise preparing the image data in a useful manner. This step 730 may also include any and all steps, methods, and procedures discussed in and regarding FIG. 6.

Step 735 includes the step of outputting the processed image data. This may include formatting, compressing, or otherwise modifying the processed image data for the purposes of interfacing with a standard display interface (e.g. VGA, DVI, HDMI, s-video, or other display interfaces). This may include, for example, the step of digital to analog conversion. This may further include transmitting the processed image data to a display driver (e.g., display driver 660). This may include, for example, providing the processed image data for display on a stand-alone display monitor, a monitor integrated into another device (for example, camera 12 or console 40), a storage device, recording device, or other destination of processed image data.

In addition to the steps listed above, method 700 may also include various wrap-up or wind-down steps, including writing updated camera use information to memory stored within camera 12 or console 40 before, during, or after any steps (for example, time of use, saved settings, white balance, preferred settings, method of use, total amount of data captured, error data, flags, temperature of device, an indication of overall camera quality or wear, identifying patient data, patient health data, user data, and other camera use or event data), sterilizing components of system 10, decoupling the components of system 10, deactivating the components, and other wrap-up steps.

The aforementioned steps for using 700 may also include the step of utilizing gathered data (including image data) to perform a medical procedure on a human or animal subject. This may include, for example, visualizing an internal bodily organ during laparoscopic surgery, or visualizing an obstruction, object, or portion of an internal bodily lumen (for example, ureteral stones). The collected data may be used to facilitate imaging and navigation of a working channel, which may include guiding disposable baskets, graspers, lasers, and other medical tools to a location of interest to enable a surgeon, doctor, nurse, or other healthcare profession to perform a surgery, operation, or procedure.

While this disclosure describes exemplary embodiments of the invention, various changes can be made and equivalents may be substituted without departing from the spirit and scope thereof. Modifications can also be made to adapt these teachings to different situations and applications, and to the use of other materials and methods, without departing from the essential scope of the invention. The invention is thus not limited to the particular examples that are disclosed, and encompasses all of the embodiments falling within the subject matter of the appended claims.

We claim:

1. A fiber optic camera system, comprising:
a fiber optic camera comprising:
an elongate sheath having a proximal end and a distal end, wherein the sheath contains:
one or more illumination optical fibers; and
an imaging bundle comprising at least one fiber optic clad and multiple fiber optic cores;
a camera body fixedly attached to the proximal end of the elongate sheath, wherein the camera body contains:
an imaging sensor optically coupled to a proximal end of the imaging bundle and configured to generate image data; and
an illumination source optically coupled to proximal ends of the illumination fibers; and
a video processing console coupled with the camera body to process the image data from the imaging sensor to generate at least one output signal,
wherein the camera body has no connection member for connecting a secondary illumination source to the camera.

2. The system of claim 1, further comprising a cable for connecting the camera body with the video processing console, wherein connection between the camera body and the video processing console is achieved solely via the cable.

3. The system of claim 1, wherein the sheath comprises polytetrafluoroethylene.

4. The system of claim 1, wherein the sheath comprises at least one of a reinforced configuration, a braided configuration or a coiled configuration.

5. The system of claim 1, wherein the camera body further contains a data serializer, and wherein the console comprises a data deserializer.

6. The system of claim 5, wherein the imaging sensor is configured to output image data using multiple parallel signals, the data serializer is configured to convert the multiple parallel signals into at least one pair of differential signals, and the deserializer is configured to convert the at least one pair of differential signals into multiple parallel signals.

7. The system of claim 1, wherein the illumination fibers include cores and clads, and wherein distal ends of the cores of the illumination fibers have a total surface area of less than about 0.000045 square-inches.

8. The system of claim 1, wherein the one or more illumination optical fibers comprise about 20 to about 40 illumination fibers.

9. The system of claim 1 wherein the imaging sensor has a responsiveness of at least 4.8V/lux-s.

10. The system of claim 1, wherein the sheath has an outer diameter of no greater than approximately 0.7 millimeters.

11. The system of claim 1, further comprising a medical device having a lumen capable of removably receiving the sheath.

12. The system of claim 11, wherein the medical device is configured for use in a urinary tract of a human or animal subject.

13. The system of claim 11, wherein a proximal end of the medical device includes a mating feature configured to mate with a corresponding mating feature on the camera body.

14. The system of claim 13, wherein the mating feature and the corresponding mating feature comprise locking features for removably coupling the medical device with the camera body.

15. The system of claim 14, wherein the locking features allow a connection between the mating feature and the corresponding mating feature to be slidably adjusted to ensure alignment within approximately 0.5 mm.

16. The system of claim 13, wherein the camera body further comprises a mechanism configured to identify the medical device and determine whether the medical device is compatible with the camera.

17. The system of claim 1, wherein the camera body further contains a one or more proximal lenses.

18. The system of claim 1, wherein the camera body further comprises a thermal bridge that thermally couples the illumination source to the camera body.

19. The system of claim 1, wherein the camera body is substantially hermetically sealed.

20. The system of claim 1, wherein the camera body further contains a nonvolatile memory module coupled with the console.

21. The system of claim 1, wherein a single control bus is electrically coupled to at least two of the imaging sensor, a nonvolatile memory module, and a circuit for controlling the illumination source.

22. The system of claim 1, further comprising a video monitor for connecting with the video processing console, wherein the output signal from the video processing console drives the video monitor.

23. The system of claim 1, wherein the illumination source includes a light emitting diode.
